(19) United States
(12) Patent Application Publication
(10) Pub. No.: US 2026/0151341 A1
(43) Pub. Date: Jun. 4, 2026

(54) NOVEL AMORPHOUS ACTIVE PHARMACEUTICAL INGREDIENTS

(71) Applicant: DISRUPTIVE PHARMA AB, Uppsala (SE)

(72) Inventors: Peter Åsberg, Stockholm (SE); Jonas Fagerberg, Uppsala (SE); Caroline Ivarsson, Tierp (SE)

(21) Appl. No.: 19/461,049

(22) Filed: Jan. 27, 2026

Related U.S. Application Data
(63) Continuation of application No. 17/923,332, now abandoned.

(30) Foreign Application Priority Data
May 6, 2020 (SE) 2050519-4

(51) Int. Cl. A61K 9/14; A61K 9/00; A61K 9/20; A61K 9/48; A61K 31/4025; A61K 31/4035; A61K 31/4174; A61K 31/4184; A61K 31/47; A61K 31/505; A61K 31/517; A61K 31/519; A61K 31/52

(52) U.S. Cl. CPC A61K 9/143; A61K 9/0053; A61K 9/2072; A61K 9/2095; A61K 9/4808; A61K 31/4025; A61K 31/4035; A61K 31/4174; A61K 31/4184; A61K 31/47; A61K 31/505; A61K 31/517; A61K 31/519; A61K 31/52

(57) ABSTRACT

The present invention is directed to a solid and substantially amorphous active pharmaceutical ingredient, to an oral pharmaceutical formulation comprising said substantially amorphous active pharmaceutical ingredient, as well as to a method for the manufacture of the same. The invention is also directed to the use of a particulate anhydrous and substantially amorphous mesoporous magnesium carbonate (MMC) to stabilize an active pharmaceutical ingredient (API).

### CROSS-REFERENCE TO RELATED APPLICATIONS

This application is a continuation of U.S. application Ser. No. 17/923,332, filed on Nov. 2, 2022, which is the 35 U.S.C. § 371 national stage filing of International Application No. PCT/EP2021/061816 filed on May 5, 2021, which in turn claims priority to Sweden Patent Application No. 2050519-4, filed May 6, 2020. The entire contents of each of the foregoing applications are included herein by reference in their entirety.

### FIELD OF THE INVENTION

The present invention is directed to a solid and substantially amorphous active pharmaceutical ingredient selected from an API such as apremilast, binimetinib, cabozantinib, eliglustat, enzalutamide, etravirine, idelalisib, tofacitinib and vandetanib, to an oral pharmaceutical formulation comprising said substantially amorphous active pharmaceutical ingredient, as well as to a method for the manufacture of the same. The invention is also directed to the use of a particulate anhydrous and substantially amorphous mesoporous magnesium carbonate (MMC) to stabilize an active pharmaceutical ingredient (API) selected from an API such as apremilast, binimetinib, cabozantinib, eliglustat, enzalutamide, etravirine, idelalisib, tofacitinib and vandetanib, into a solid and substantially amorphous compound form.

### BACKGROUND

One of the largest challenges in pharmaceutical drug development is that drug substances (Active Pharmaceutical Ingredients, API's) often are insoluble, or poorly soluble, in aqueous media. Insufficient or poor API solubility (drug solubility) provides insufficient or poor bioavailability, which in turn typically means poor plasma exposure of a drug product when administered to subjects such as humans and animals. Also fasted or fed conditions may result in significant variability in drug exposure between patients as well as variability in drug exposure for a single patient.

A drug substance is mostly used in its' crystalline form when formulated into pharmaceutical products (drug products). Crystalline forms of poorly soluble drugs have solubility limited absorption, whereas amorphous forms of drug substances provide a better solubility and dissolution rate than the corresponding crystal form. However, a problem with amorphous forms of drugs is that they almost always lack storage stability.

Approximately 40 percent of the APIs currently on the market and 70 percent of all APIs in development phase, suffer from poor aqueous solubility, as defined by the Biopharmaceutics Classification System (BCS). Consequently, problems with poorly soluble APIs already being commercialized, as well as API's in research and development pipelines, are significant. However, exact numbers differ slightly from one reference to another. Due to their challenging solubility limitations, APIs in pharmaceutical development may never reach the market for this reason.

Various formulation techniques for improving drug solubility exist, such as particle size reduction, formation of cyclodextrin complexes and amorphous formulations. The aim with these technologies is to increase the bioavailability for orally delivered drugs suffering from poor drug solubility properties. However, enhancing the aqueous solubility of orally administered drugs is a challenge in pharmaceutical drug development, since many of these techniques are expensive and/or still provides insufficient solubility improvement. Furthermore, long term stability in the amorphous phase, with only minor or no amounts of the API in crystalline form, continues to be a problem also with the currently existing techniques used in pharmaceutical formulation development.

Gupte et al. (British Journal of Pharmaceutical Research 16 (6), 2017, 1-9) disclose mesoporous silica and the loading of the silica with APIs such as ibuprofen, itraconazole, and telmisartan. Gupte refers to a study where the silica is reported to stabilize itraconazole at a loading of more than 32% by weight.

WO2017/174458 discloses a mesoporous magnesium carbonate material which is stated to allow an API load of up to 60% by weight of itraconazole by soaking. Itraconazole is kept amorphous at an API load of up to 30% by weight.

Yang et al. (International Journal of Pharmaceutics 525, 2017, 183-190) disclose a mesoporous magnesium carbonate material (particle size<50 μm) comprising the APIs tolfenamic acid and rimonabant.

WO 2020/096513 discloses a solid and substantially amorphous active pharmaceutical ingredient, an oral pharmaceutical formulation comprising a substantially amorphous active pharmaceutical ingredient and a method for the manufacture of the same. Also disclosed therein, is a particulate anhydrous and substantially amorphous mesoporous magnesium carbonate (MMC) and the use of said MMC to stabilize an active pharmaceutical ingredient (API).

### DESCRIPTION OF THE INVENTION

A problem underlying the present invention is to provide a solid substantially amorphous active pharmaceutical ingredient (API), which is maintained in essentially amorphous form during storage. More specifically, the API may be selected from any one of apremilast, binimetinib, cabozantinib, eliglustat, enzalutamide, etravirine, idelalisib, tofacitinib and vandetanib. Any such API when used in accordance with the present invention, may be used in its free form, in acid or neutral form, in salt form, in crystalline or polymorph form, or as a solvate or a hydrate.

Yet a problem underlying the invention is to provide an oral pharmaceutical formulation (drug product) enabling a therapeutically sufficient API load without having to increase the size of capsules and/or tablets, which often is a concern to patients with problems to swallow too large capsules or tablets. Hence, patient compliance is a further aspect of the invention. Usually, the API load in combination with the size of a tablet or capsule is a limiting factor.

One aspect of the present invention is a solid substantially amorphous active pharmaceutical ingredient, comprising an API in an amount of at least 20% by weight, in admixture with a particulate anhydrous and substantially amorphous mesoporous magnesium carbonate (MMC) which MMC may optionally have been heat treated, and wherein said MMC has:
(i) pores with a peak pore width in the range of 2 nm to 10 nm;
(ii) an average BET surface area in the range of 250-900 m²/g;
(iii) an average pore volume in the range of 0.5-1.2 cm³/g; and
(iv) an average particle size distribution exhibiting a d₁₀ value of 70-430 μm.

APIs useful in accordance with the present invention may be selected from any one of apremilast (such as the NDA approved drug Otezla®); binimetinib (such as the NDA approved drug Mektovi®); cabozantinib (such as the NDA approved drug Cabometyx®); eliglustat (such as the NDA approved drug Cerdelga®); enzalutamide (such as the NDA approved drug Xtandi®); etravirine (such as the NDA approved drug Intelence®); idelalisib (such as the NDA approved drug Zydelig®); tofacitinib (such as the NDA approved drug Xeljanz®); and vandetanib (such as the NDA approved drug Caprelsa®). Any such API when used in accordance with the present invention, may be used in its free form, in acid or neutral form, in salt form, in crystalline or polymorph form, or as a solvate or a hydrate.

Yet further examples of APIs which may be useful in accordance with the invention are empagliflozin (such as the NDA approved drug Jardiance®); sitagliptin phosphate (such as the NDA approved drug Januvia®); canagliflozin (such as the NDA approved drug Invokana®); empagliflozin, linagliptin (such as the NDA approved drug Glyxambi®); linagliptin (such as the NDA approved drug Tradjenta®); alogliptin benzoate (such as the NDA approved drug Nesina®); Omarigliptin (such as the NDA approved drug Marizev®); Saxagliptin hydrochloride (such as the NDA approved drug Onglyza®); Saxagliptin Dapagliflozin (such as the NDA approved drug SaxaDapa®); Alectinib hydrochloride (such as the NDA approved drug Alecensa®); Dabrafenib mesylate (such as the NDA approved drug Tafinlar®); Ceritinib (such as the NDA approved drug Zykadia®); Lorlatinib (such as the NDA approved drug Lorbrena®); Capmatinib (such as the development compound INC280); Selpercatinib (such as the development compound LOXO-292); Venetoclax (such as the NDA approved drug Venclexta®); Treprostinil diolamine (such as the NDA approved drug Orenitram®); Ralinepag (such as the development compound APD811); or Remdesivir (such as the development compound GS-5734). Any such API when used in accordance with the present invention, may be used in its free form, in acid or neutral form, in salt form, in crystalline or polymorph form, or as a solvate or a hydrate.

An aspect of the present invention is a solid substantially amorphous active pharmaceutical ingredient, comprising an API in an amount of at least 20% by weight, in admixture with a particulate anhydrous and substantially amorphous mesoporous magnesium carbonate (MMC), wherein said MMC has:
(i) pores with a peak pore width in the range of 2 nm to 10 nm;
(ii) an average BET surface area in the range of 250-900 m²/g;
(iii) an average pore volume in the range of 0.5-1.2 cm³/g; and
(iv) an average particle size distribution exhibiting a d₁₀ value of 70-430 μm;
   and wherein the API is selected from any one of apremilast, binimetinib, cabozantinib, eliglustat, enzalutamide, etravirine, idelalisib, tofacitinib and vandetanib.

Yet an aspect of the present invention is a solid substantially amorphous active pharmaceutical ingredient, comprising an API in an amount of at least 20% by weight, in admixture with a heat-treated particulate anhydrous and substantially amorphous mesoporous magnesium carbonate (MMC), wherein said MMC has:

(i) pores with a peak pore width in the range of 2 nm to 10 nm;

(ii) an average BET surface area in the range of 250-900 m²/g;

(iii) an average pore volume in the range of 0.5-1.2 cm³/g; and

(iv) an average particle size distribution exhibiting a d₁₀ value of 70-430 μm;

and wherein the API is selected from any one of apremilast, binimetinib, cabozantinib, eliglustat, enzalutamide, etravirine, idelalisib, tofacitinib and vandetanib.

Yet an aspect of the invention is a solid substantially amorphous active pharmaceutical ingredient, comprising an API in an amount of at least 20% by weight, in admixture with a particulate anhydrous and substantially amorphous mesoporous magnesium carbonate (MMC), wherein said MMC has:
(i) pores with a peak pore width in the range of 2 nm to 10 nm;
(ii) an average BET surface area in the range of 250-900 m²/g;
(iii) an average pore volume in the range of 0.5-1.0 cm³/g; and
(iv) an average particle size distribution exhibiting a d₁₀ value of 70-430 μm;
   and wherein the API is selected from any one of apremilast, binimetinib, cabozantinib, eliglustat, enzalutamide, etravirine, idelalisib, tofacitinib and vandetanib.

Yet an aspect of the invention is a solid substantially amorphous active pharmaceutical ingredient, comprising an API in an amount of at least 20% by weight, in admixture with a heat-treated particulate anhydrous and substantially amorphous mesoporous magnesium carbonate (MMC), wherein said MMC has:
(i) pores with a peak pore width in the range of 2 nm to 10 nm;
(ii) an average BET surface area in the range of 250-900 m²/g;
(iii) an average pore volume in the range of 0.5-1.0 cm³/g; and
(iv) an average particle size distribution exhibiting a d₁₀ value of 70-430 μm;
   and wherein the API is selected from any one of apremilast, binimetinib, cabozantinib, eliglustat, enzalutamide, etravirine, idelalisib, tofacitinib and vandetanib.

Yet an aspect of the invention is a solid substantially amorphous active pharmaceutical ingredient, comprising an API in an amount of at least 20% by weight, in admixture with a particulate anhydrous and substantially amorphous mesoporous magnesium carbonate (MMC), wherein said MMC has:
(i) pores with a peak pore width in the range of 2 nm to 10 nm;
(ii) an average BET surface area in the range of 250-900 m²/g;
(iii) an average pore volume in the range of 0.5-0.9 cm³/g; and
(iv) an average particle size distribution exhibiting a d₁₀ value of 70-430 μm;
   and wherein the API is selected from any one of apremilast, binimetinib, cabozantinib, eliglustat, enzalutamide, etravirine, idelalisib, tofacitinib and vandetanib.

Yet an aspect of the invention is a solid substantially amorphous active pharmaceutical ingredient, comprising an API in an amount of at least 20% by weight, in admixture with a heat-treated particulate anhydrous and substantially amorphous mesoporous magnesium carbonate (MMC), wherein said MMC has:
(i) pores with a peak pore width in the range of 2 nm to 10 nm;
(ii) an average BET surface area in the range of 250-900 m²/g;
(iii) an average pore volume in the range of 0.5-0.9 cm³/g; and
(iv) an average particle size distribution exhibiting a d₁₀ value of 70-430 μm;
   and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein, wherein the particulate anhydrous and substantially amorphous mesoporous magnesium carbonate (MMC) further comprises magnesium oxide (MgO).

In yet an aspect of the invention, a particulate anhydrous and substantially amorphous mesoporous magnesium carbonate (MMC) as used in accordance with the invention, has pores with a peak pore width in the range of 3 nm to 8 nm.

In yet an aspect of the invention, a particulate anhydrous and substantially amorphous mesoporous magnesium carbonate (MMC) as used in accordance with the invention, has pores with a peak pore width in the range of 4 nm to 7 nm.

In yet an aspect of the invention, a particulate anhydrous and substantially amorphous mesoporous magnesium carbonate (MMC) as used in accordance with the invention, has an average BET surface area in the range of 250-800 m²/g.

In yet an aspect of the invention, a particulate anhydrous and substantially amorphous mesoporous particulate magnesium carbonate (MMC) as used in accordance with the invention, has an average BET surface area in the range of 300-700 m²/g.

In yet an aspect of the invention, a particulate anhydrous and substantially amorphous mesoporous magnesium carbonate (MMC) as used in accordance with the invention, has an average pore volume in the range of 0.5-1.0 cm³/g.

In yet an aspect of the invention, a particulate anhydrous and substantially amorphous mesoporous magnesium carbonate (MMC) as used in accordance with the invention, has an average pore volume in the range of 0.5-0.9 cm³/g.

Yet an aspect of the invention, is a solid substantially amorphous active pharmaceutical ingredient as disclosed and claimed herein (MMC-API), wherein the amount of API is at least 25% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the amount of API is at least 30% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the amount of API is at least 35% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the amount of API is at least 40% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the amount of API is at least 45% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient s disclosed and claimed herein (MMC-API), wherein the amount of API is at least 50% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the amount of API is at least 55% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the amount of API is at least 60% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the amount of API is at least 65% by weight and wherein said API is selected from any one of apremilast, binimetinib, cabozantinib, eliglustat, enzalutamide, etravirine, idelalisib, tofacitinib and vandetanib.

Yet an aspect of the invention is a solid substantially amorphous active pharmaceutical ingredient as disclosed and claimed herein (MMC-API), comprising an API in an amount of 20-60% by weight and wherein said API is selected from any one of apremilast, binimetinib, cabozantinib, eliglustat, enzalutamide, etravirine, idelalisib, tofacitinib and vandetanib.

Yet an aspect of the invention is a solid substantially amorphous active pharmaceutical ingredient as disclosed and claimed herein (MMC-API), comprising an API in an amount of 30-50% by weight and wherein said API is selected from any one of apremilast, binimetinib, cabozantinib, eliglustat, enzalutamide, etravirine, idelalisib, tofacitinib and vandetanib.

Yet an aspect of the invention is a solid substantially amorphous active pharmaceutical ingredient as disclosed and claimed herein (MMC-API), comprising an API in an amount of 35-50% by weight and wherein said API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein said substantially amorphous active ingredient (MMC-API) has a compressibility index of 15 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein said substantially amorphous active ingredient (MMC-API) has a compressibility index of 12 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein said substantially amorphous active ingredient (MMC-API) has a compressibility index of 10 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein said substantially amorphous active ingredient (MMC-API) has a compressibility index of 9 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein said substantially amorphous active ingredient (MMC-API) has a compressibility index of 8 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein said substantially amorphous active ingredient (MMC-API) has a Hausner ratio of 1.18 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein said substantially amorphous active ingredient (MMC-API) has a Hausner ratio of 1.15 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the Hausner ratio is 1.14 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the Hausner ratio is 1.13 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the Hausner ratio is 1.12 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the Hausner ratio is 1.11 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), wherein the Hausner ratio is 1.10 or less, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), comprising a particulate anhydrous mesoporous magnesium carbonate (MMC) having an average particle size distribution exhibiting a d₁₀ value of 80 μm or higher, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), comprising a particulate anhydrous mesoporous magnesium carbonate (MMC) having an average particle size distribution exhibiting a d₁₀ value of 90 μm or higher, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), comprising a particulate anhydrous mesoporous magnesium carbonate (MMC) having an average particle size distribution exhibiting a d₁₀ value of 70-300 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), comprising an anhydrous mesoporous particulate magnesium carbonate (MMC) having an average particle size distribution exhibiting a d₅₀ value of 75-500 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), comprising a particulate anhydrous mesoporous magnesium carbonate (MMC) having an average particle size distribution exhibiting a d₅₀ value of 75-350 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), comprising a particulate anhydrous mesoporous magnesium carbonate (MMC) having an average particle size distribution exhibiting a doo value of 170-500 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient as disclosed and claimed herein (MMC-API), comprising a particulate anhydrous mesoporous magnesium carbonate (MMC) having an average particle size distribution exhibiting a doo value of 210-450 μm, and wherein the API is selected from any one of apremilast, binimetinib, cabozantinib, eliglustat, enzalutamide, etravirine, idelalisib, tofacitinib and vandetanib.

A further aspect of the invention, is an oral pharmaceutical formulation, comprising a solid amorphous active pharmaceutical ingredient as disclosed and claimed herein (MMC-API), wherein the API is selected from any one of apremilast, binimetinib, cabozantinib, eliglustat, enzalutamide, etravirine, idelalisib, tofacitinib and vandetanib;
in admixture with a pharmaceutically and pharmacologically acceptable excipient, carrier, and/or diluent.

Yet an aspect of the invention, is an oral pharmaceutical formulation comprising a solid substantially amorphous active pharmaceutical ingredient as disclosed and claimed herein (MMC-API), which is bioequivalent to a formulation comprising an API which has been approved (NDA approved) by a medical authority such as the FDA (Food and Drug Administration) in the US, or EMA (European Medicines Agency) in the EU.

Yet an aspect of the invention, is the use of a particulate anhydrous and substantially amorphous mesoporous magnesium carbonate (MMC) as disclosed herein, for use in stabilizing an active pharmaceutical ingredient (API) selected from any one of apremilast, binimetinib, cabozantinib, eliglustat, enzalutamide, etravirine, idelalisib, tofacitinib and vandetanib.

In an aspect of the present invention, a particulate anhydrous and substantially amorphous mesoporous magnesium carbonate (MMC) as used in accordance with the invention, has:
(i) pores with a peak pore width in the range of 2 nm to 10 nm;
(ii) an average BET surface area in the range of 250-900 m²/g;
(iii) an average pore volume in the range of 0.5-1.2 cm³/g; and
(iv) an average particle size distribution exhibiting a d₁₀ value of 70-430 μm.

In a further aspect of the invention, the particulate anhydrous and substantially amorphous mesoporous magnesium carbonate (MMC) as used in accordance with the invention, may further comprise magnesium oxide (MgO).

In yet an aspect of the invention, the particulate anhydrous and substantially amorphous mesoporous magnesium carbonate (MMC) as used in accordance with the invention, has been heat treated, providing a heat treated particulate anhydrous and substantially amorphous mesoporous magnesium carbonate (MMC).

In yet an aspect of the invention, the particulate anhydrous and substantially amorphous mesoporous magnesium carbonate (MMC) as used in accordance with the invention, has a compressibility index of 15 or less.

In yet an aspect of the invention, the particulate anhydrous and substantially amorphous mesoporous magnesium carbonate (MMC) as used in accordance with the invention, has a compressibility index of 12 or less.

In yet an aspect of the invention, the particulate anhydrous and substantially amorphous mesoporous magnesium carbonate (MMC) as used in accordance with the invention, has a compressibility index of 10 or less.

In yet an aspect of the invention, the particulate anhydrous and substantially amorphous mesoporous particulate magnesium carbonate (MMC) as used in accordance with the invention, has a compressibility index of 9 or less.

In yet an aspect of the invention, the particulate anhydrous and substantially amorphous mesoporous magnesium carbonate (MMC) as used in accordance with the invention, has a compressibility index of 8 or less.

In yet an aspect of the invention, the particulate anhydrous and substantially amorphous mesoporous magnesium carbonate (MMC) as used in accordance with the invention, has a Hausner ratio of 1.18 or less.

In yet an aspect of the invention, the particulate anhydrous and substantially amorphous mesoporous magnesium carbonate (MMC) as used in accordance with the invention, has a Hausner ratio of 1.15 or less.

In yet an aspect of the invention, the particulate anhydrous and substantially amorphous mesoporous magnesium carbonate (MMC) as used in accordance with the invention, has a Hausner ratio of 1.14 or less.

In yet an aspect of the invention, the particulate anhydrous and substantially amorphous mesoporous magnesium carbonate (MMC) as used in accordance with the invention, has a Hausner ratio of 1.13 or less.

In yet an aspect of the invention, the particulate anhydrous and substantially amorphous mesoporous magnesium carbonate (MMC) as used in accordance with the invention, has a Hausner ratio of 1.12 or less.

In yet an aspect of the invention, the particulate anhydrous and substantially amorphous mesoporous magnesium carbonate (MMC) as used in accordance with the invention, has a Hausner ratio of 1.11 or less.

In yet an aspect of the invention, the particulate anhydrous and substantially amorphous mesoporous magnesium carbonate (MMC) as used in accordance with the invention, has a Hausner ratio of 1.10 or less.

In yet an aspect of the invention, the particulate anhydrous and substantially amorphous mesoporous magnesium carbonate (MMC) as used in accordance with the invention, has an average particle size distribution exhibiting a d₁₀ value of 70 μm or higher.

In yet an aspect of the invention, the particulate anhydrous and substantially amorphous mesoporous magnesium carbonate (MMC) as used in accordance with the invention, has an average particle size distribution exhibiting a d₁₀ value of 80 μm or higher.

In yet an aspect of the invention, the particulate anhydrous and substantially amorphous mesoporous magnesium carbonate (MMC) as used in accordance with the invention, has an average particle size distribution exhibiting a d₁₀ value of 90 μm or higher.

In yet an aspect of the invention, the particulate anhydrous and substantially amorphous mesoporous magnesium carbonate (MMC) as used in accordance with the invention, has an average particle size distribution exhibiting a d₁₀ value of 100 μm or higher.

In yet a further aspect of the invention, the particulate anhydrous and substantially amorphous mesoporous magnesium carbonate (MMC) as used in accordance with the invention, has an average particle size distribution exhibiting a d₁₀ value of 70-300 μm.

In yet a further aspect of the invention, the particulate anhydrous and substantially amorphous mesoporous magnesium carbonate (MMC) as used in accordance with the invention, has an average particle size distribution exhibiting a d₅₀ value of 75-500 μm.

In yet a further aspect of the invention, the particulate anhydrous and substantially amorphous mesoporous magnesium carbonate (MMC) as used in accordance with the invention, has an average particle size distribution exhibiting a d₅₀ value of 75-350 μm.

In yet an aspect of the invention, the particulate anhydrous and substantially amorphous mesoporous magnesium carbonate (MMC) as used in accordance with the invention, has an average particle size distribution exhibiting a deo value of 170-500 μm.

In yet an aspect of the invention, the particulate anhydrous and substantially amorphous mesoporous magnesium carbonate (MMC) as used in accordance with the invention, has an average particle size distribution exhibiting a deo value of 210-450 μm.

In one aspect of the present invention, the particulate anhydrous and substantially amorphous mesoporous magnesium carbonate (MMC) as used in accordance with the invention, is heat treated MMC.

One aspect of the invention is a solid substantially amorphous active pharmaceutical ingredient, comprising an API in an amount of at least 20% by weight, in admixture with a particulate anhydrous and substantially amorphous mesoporous magnesium carbonate (MMC), wherein said admixture of MMC and API (MMC-API) has:
(i) pores with a peak pore width in the range of 2 nm to 10 nm;
(ii) an average BET surface area in the range of 150-600 m²/g;
(iii) an average pore volume in the range of 0.1-1.2 cm³/g; and
(iv) an average particle size distribution exhibiting a d₁₀ value of 70-430 μm;
   and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having pores with a peak pore width in the range of 3 nm to 9 nm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having pores with a peak pore width in the range of 3 nm to 8 nm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having pores with a peak pore width in the range of 3 nm to 7 nm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average BET surface area in the range of 150-500 m²/g, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average BET surface area in the range of 60-430 m²/g, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average pore volume in the range of 0.1-0.9 cm³/g, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average pore volume in the range of 0.1-0.8 cm³/g, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average particle size distribution exhibiting a d₁₀ value of 70-350 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average particle size distribution exhibiting a d₅₀ value of 75-500 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average particle size distribution exhibiting a d₅₀ value of 75-400 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average particle size distribution exhibiting a deo value of 170-500 μm, and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient (MMC-API admixture) as herein described and claimed, having an average particle size distribution exhibiting a do value of 220-500 μm, and wherein the API is selected from any one of apremilast, binimetinib, cabozantinib, eliglustat, enzalutamide, etravirine, idelalisib, tofacitinib and vandetanib.

One aspect of the invention, is a solid substantially amorphous active pharmaceutical ingredient, comprising an API in an amount of at least 20% by weight, in admixture with a particulate anhydrous and substantially amorphous mesoporous magnesium carbonate (MMC), wherein said admixture of MMC-API (MMC-API admixture) has:
(i) pores with a peak pore width in the range of 2 nm to 10 nm;
(ii) an average BET surface area in the range of 150-600 m²/g;
(iii) an average pore volume in the range of 0.1-1.2 cm³/g; and
(iv) an average particle size distribution exhibiting a d₁₀ value of 70-430 μm; and
(v) an average particle size distribution exhibiting a d₅₀ value of 75-500 μm;
   and wherein the API is selected from any one of apremilast, binimetinib, cabozantinib, eliglustat, enzalutamide, etravirine, idelalisib, tofacitinib and vandetanib.

Yet an aspect of the invention, is a solid substantially amorphous active pharmaceutical ingredient, comprising an API in an amount of at least 20% by weight, in admixture with a particulate anhydrous and substantially amorphous mesoporous magnesium carbonate (MMC), and wherein said admixture of MMC and API (MMC-API admixture) has:
(i) pores with a peak pore width in the range of 2 nm to 10 nm;
(ii) an average BET surface area in the range of 150-600 m²/g;
(iii) an average pore volume in the range of 0.1-1.2 cm³/g; and
(iv) an average particle size distribution exhibiting a d₁₀ value of 70-430 μm;
(v) an average particle size distribution exhibiting a d₅₀ value of 75-500 μm;
(vi) an average particle size distribution exhibiting a deo value of 170-500 μm;
   and wherein the API is selected from any one of apremilast, binimetinib, cabozantinib, eliglustat, enzalutamide, etravirine, idelalisib, tofacitinib and vandetanib.

High substance load (i.e. API load) may often lead to crystallization, especially for substances (APIs) with high tendency to crystallize. The present inventors have shown that a particulate anhydrous and substantially amorphous mesoporous magnesium carbonate (MMC) as used in accordance with the invention, may be useful even at high API load of an active pharmaceutical ingredient (API) described herein, to provide a substance (API) which remains XRPD amorphous as well as DSC amorphous during storage.

Yet an aspect of the invention, is a method for the manufacture of a solid substantially amorphous active pharmaceutical ingredient (MMC-API), comprising the steps of:
a. dissolving an API in a solvent such as an organic solvent;
b. adding a particulate anhydrous and substantially amorphous mesoporous magnesium carbonate (MMC) as disclosed and claimed herein, to the API solution of step (a);
c. evaporating the solvent; and
d. optionally drying the final product;
   and wherein the API is selected from any one of apremilast, binimetinib, cabozantinib, eliglustat, enzalutamide, etravirine, idelalisib, tofacitinib and vandetanib.

### Definitions

As used herein, the terms “poorly soluble API”, “poorly soluble drug” and “insufficient drug solubility” refers to an API that requires more than 250 ml aqueous media in order to dissolve at a pH of from about 1 to about 8, based on the highest dose strength of an immediate release product, as defined for BCS Class II drugs.

As used herein the term “amorphous API” and “substantially amorphous API” is defined as a solid active pharmaceutical ingredient which is maintained in its amorphous form and is substantially free from crystalline material as detected by X-ray powder diffraction (XRPD) and differential scanning calorimetry (DSC). An amorphous API, or a substantially amorphous API, is thus substantially free of crystalline material and without sharp peaks in an XRPD diffractogram, and also lacks a melting point endotherm as measured by DSC.

As used herein the term “stable API” refers to an API which is physically stable and which continues to exist in a substantially amorphous form under storage conditions such as at room temperature (18-25° C.) and at a relative humidity of 25%, or at room temperature (18-25° C.) and at a relative humidity of 75%, during at least a month, or during at least 3 months, or during at least 6 months, 9 months or up to at least one year or longer. During storage under these conditions, there is no presence of crystals or substantially no presence of crystals, as detected by XRPD and by DSC.

As used herein, the term “crystalline API” is defined as an API where the structural units are arranged in fixed geometric pattern or lattices, so that crystalline solids have rigid long-range order. The structural units that constitute the crystal may be atoms, molecules or ions. Crystalline solid material shows definitive melting points and displays sharp characteristic crystalline peaks in an XRPD diffractogram (XRPD pattern).

As used herein, the wording “MMC” means a particulate anhydrous and substantially amorphous mesoporous magnesium carbonate (MMC), which may or may not contain residual magnesium oxide and/or methanol.

As used herein, the wording “MMC-API” is a solid substantially amorphous active pharmaceutical ingredient, comprising an API in an amount of at least 20% by weight, in admixture with a particulate anhydrous and substantially amorphous mesoporous magnesium carbonate (MMC) as herein claimed and described.

Whenever the wording “solid substantially amorphous active pharmaceutical ingredient” and/or “MMC-API” is used throughout the present specification and claims, it means an API in admixture with a particulate and substantially amorphous mesoporous magnesium carbonate (MMC). When an API and particulate anhydrous and substantially amorphous mesoporous magnesium carbonate (MMC) of the invention is brought together as an admixture, it provides a solid substantially amorphous active pharmaceutical ingredient (MMC-API).

The wording “an API in admixture with a particulate anhydrous and substantially amorphous mesoporous magnesium carbonate (MMC)” is defined as an API starting material which has been brought into admixture with a particulate anhydrous and substantially amorphous mesoporous particulate magnesium carbonate (MMC),

providing an API loaded into MMC (herein referred to as “MMC-API” or “MMC-API admixture”), according to the present invention.

As used herein, the wording “API loaded into MMC”, means an API in admixture with MMC (i.e. a particulate anhydrous and substantially amorphous mesoporous particulate magnesium carbonate).

As used herein, the term “API load” refers to the amount of API starting material that can be included in a solid substantially amorphous active pharmaceutical ingredient as herein described and claimed (MMC-API or MMC-API admixture), or as formulated into a pharmaceutical formulation (i.e. the amount of API in the drug product).

As used herein, the wording “high API load” is defined as an amount of at least 20% by weight of an API used as starting material, which may be brought into admixture with a particulate anhydrous and substantially amorphous mesoporous magnesium carbonate (MMC) as disclosed and claimed herein, providing a solid and substantially amorphous active pharmaceutical ingredient (API) (MMC-API).

As used herein, the abbreviation “wt %” means % by weight, which is the weight fraction expressed in percent of a component in relation to the total weight of a mixture, as herein described and claimed.

As used herein, the wording “stabilizing” is defined as the use of a particulate anhydrous and substantially amorphous mesoporous magnesium carbonate (MMC) as herein described and claimed, which when brought together as an admixture with an active pharmaceutical ingredient as starting material, provides a solid substantially amorphous active pharmaceutical ingredient (MMC-API) which is maintained in amorphous form during at least 1 month storage at room temperature and 75% relative humidity.

As used herein, the wording “peak pore width” correspond to the pore width value on the x-axis extracted from the maximum (peak) value of incremental pore volume on the y-axis in a pore size distribution curve obtained from nitrogen gas adsorption.

As used herein, the wording “room temperature” or “RT” is defined as a temperature of 18-25° C.

As used herein, the term “pharmaceutical formulation” refers to a pharmaceutical formulation in which an active pharmaceutical ingredient has been formulated into a drug product.

As used herein, the term “drug product” is defined as a pharmaceutical formulation comprising a solid substantially amorphous active pharmaceutical Ingredient (MMC-API) as herein described and claimed, together with a pharmaceutically and pharmacologically acceptable excipient, carrier and/or diluent.

As used herein, the term “active pharmaceutical agent (API)” refers to a substance which is the therapeutically active ingredient in a drug substance administered to humans and/or animals in need of medical therapy.

An API useful as starting material when making a substantially amorphous active pharmaceutical ingredient in accordance with the invention, may be an API in its free form, acid or neutral form, or in salt form, in crystalline or polymorph form, or as a solvate or a hydrate.

As used herein, the term “pharmaceutically and pharmacologically acceptable excipient, carrier and/or diluent” refers to any non-therapeutic agent that may be included in a pharmaceutical formulation when formulating an API to e.g. a drug product.

The terms “compressibility index” (also called “Carr index”) and “Hausner ratio” are used to predict powder flowability. The term “flowability” as used herein refers to the ability of a powder to flow. Flowability is an important factor for the process of making tablets or capsules.

As used throughout the specification and claims, the term “compressibility index” (Carr index) is a measure of bulk density, size and shape, surface area, moisture content and cohesiveness both in the context of a solid substantially amorphous active pharmaceutical ingredient (MMC-API) as herein disclosed and claimed, as well as in the context of a particulate anhydrous and substantially amorphous mesoporous magnesium carbonate (MMC) as herein disclosed and claimed, and is the ratio (in percentage) between the difference in tapped density and bulk density, and the tapped density. A lower value of the Carr index means a higher flowability, and a higher Carr index means a lower flowability.

The inventors of the present invention have realized that the particle size distribution of a particulate anhydrous and substantially amorphous mesoporous magnesium carbonate (both with and without API), affects the flowability of the particles (i.e. both with and without API).

Particles that are too small may provide good release properties but may also exhibit poor flow properties, it resembles a dust. On the other hand, too large particles tend to fall apart during API loading, leading to formation of small particles and a non-homogenous size distribution which in turn leads to low dose accuracy and small particles reducing the flowability.

As used throughout the specification and claims, the term “Hausner ratio” refers to the flowability of a solid substantially amorphous active pharmaceutical ingredient (MMC-API) as herein disclosed and claimed, as well as to the flowability of a particulate anhydrous and substantially amorphous mesoporous magnesium carbonate (MMC) as herein disclosed and claimed, and is the ratio between the tapped density and the bulk density. A lower Hausner value means a higher flowability, and a higher Hausner ratio means a lower flowability.

Flowability is classified according to European Pharmacopeia 9.0, and is expressed as excellent, good, fair, passable, poor, very poor, and very, very poor.

The bulk density of a powder is the ratio of the mass of an untapped powder sample and its volume including the contribution of the interparticulate void volume. Hence, the bulk density depends on both the density of powder particles and the spatial arrangement of particles in the powder bed. The bulk density is expressed in grams per milliliter (g/ml) although the international unit is kilogram per cubic meter (1 g/ml=1000 kg/m³) because the measurements are made using cylinders. It may also be expressed in grams per cubic centimeter (g/cm³).

By controlling the particle size, flowability of the particles may be modified or controlled by for example reducing the number of small particles.

As used herein, the term “average particle size distribution” exhibiting a d₁₀ value of 70 μm or higher, means that a maximum of 10% by volume of the particles have a particle size smaller than 70 μm.

As used herein, the term “average particle size distribution” exhibiting a d₁₀ value of 80 μm or higher, means that a maximum of 10% by volume of the particles have a particle size smaller than 80 μm.

As used herein, the term “average particle size distribution” exhibiting a d₁₀ value of 90 μm or higher, means that a maximum of 10% by volume of the particles have a particle size smaller than 90 μm or higher.

As used herein, the term “average particle size distribution” exhibiting a d₁₀ value of 100 μm or higher, means that a maximum of 10% by volume of the particles have a particle size smaller than 100 μm or higher.

As used herein, the term “average particle size distribution” exhibiting a d₁₀ value of 70-430 μm, means that 10% by volume of the particles have a particle size smaller than 70-430 μm.

As used herein, the term “average particle size distribution” exhibiting a d₁₀ value of 70-300 μm, means that 10% by volume of the particles have a particle size smaller than 70-300 μm.

As used herein, the term “average particle size distribution” exhibiting a d₅₀ value of 75-500 μm, means that 50% by volume of the particles have a particle size of 75-500 μm.

As used herein, the term “average particle size distribution” exhibiting a d₅₀ value of 75-350 μm, means that 50% by volume of the particles have a particle size of 75-350 μm.

As used herein, the term “average particle size distribution” exhibiting a deo value of 170-500 μm, means that 90% by volume of the particles have a particle size smaller than 170-500 μm.

As used herein, the term “average particle size distribution” exhibiting a deo value of 210-450 μm, means that 90% by volume of the particles have a particle size smaller than 210-450 μm.

As used herein, the term “average particle size distribution” exhibiting a d₁₀ value of 70-350 μm, means that 10% by volume of the particles have a particle size smaller than 70-350 μm.

As used herein, the term “average particle size distribution” exhibiting a d₅₀ value of 75-500 μm, means that 50% by volume of the particles have a particle size of 75-500 μm.

As used herein, the term “average particle size distribution” exhibiting a d₅₀ value of 75-400 μm, means that 50% by volume of the particles have a particle size of 75-400 μm.

As used herein, the term “average particle size distribution” exhibiting a do value of 170-500 μm, means that 90% by volume of the particles have a particle size smaller than 170-500 μm.

As used herein, the term “average particle size distribution” exhibiting a doo value of 220-500 μm, means that 90% by volume of the particles have a particle size smaller than 220-500 μm.

A particulate anhydrous and substantially amorphous mesoporous material of magnesium carbonate (MMC) as herein disclosed and claimed, consists of many particles where there may be a variability in technical parameters such as BET surface area, pore volume, and particle size distribution between particles. Whenever the term “average” is used, such as but not limited to, the terms “average BET surface area”, “average pore volume” and “average particle size”, it thus refers to the average pore size, average BET surface area, average pore volume, and average particle size for a particular particle of mesoporous magnesium carbonate (MMC) and/or MMC-API.

The wording “average” as used throughout the present specification and claims in relation to “average BET surface area”, “average pore volume”, and “average particle size distribution”, means that a given value for any such technical parameter may deviate by +/−2% of the given value, or +/−5%, or +/−10%, of the numeric values, where applicable.

In one aspect of the invention, a particulate anhydrous and substantially amorphous mesoporous material of magnesium carbonate (MMC) is amorphous as measured by X-ray powder diffraction (XRPD amorphous).

The wording “heat treated particulate anhydrous and substantially amorphous mesoporous magnesium carbonate (MMC)” as used herein, means particulate anhydrous and substantially amorphous mesoporous magnesium carbonate (MMC) which has been subjected to a heat treatment step during manufacture.

In still an aspect of the invention, a particulate anhydrous and substantially amorphous mesoporous material of magnesium carbonate (MMC) according to the present invention, is amorphous as measured by differential scanning calorimetry (DSC amorphous).

In yet an aspect of the invention, a particulate anhydrous and substantially amorphous mesoporous material of magnesium carbonate (MMC) according to the present invention is both XRPD amorphous and DSC amorphous.

In yet an aspect of the invention, a particulate anhydrous and substantially amorphous mesoporous material is a particulate composite material of X-ray amorphous and DSC amorphous mesoporous magnesium carbonate (MMC) and magnesium oxide (MgO).

In one aspect of the invention, the magnesium oxide (MgO) is a residue from the process for making a particulate anhydrous and a substantially amorphous mesoporous material as herein described and claimed.

### DESCRIPTION OF THE FIGURES

FIG. 1 illustrates the peak pore width of MMC Batch 2 compared to the API idelalisib loaded into MMC Batch 2 (referred to as MMC-idelalisib).

FIG. 2a and FIG. 2b shows thermograms recorded by using DSC for crystalline APIs (dotted shown only for A-batches for clarity), corresponding intermediates (lines) and stability samples (dashed), at T1 (dashed) (i.e. after storage for 1 month at 75% relative humidity and room temperature), T6 (dash-dotted), and T8 or T12 (dash-dot-dotted). None of MMC-APIs or stability samples exhibit endothermic signals indicating presence of crystalline API. The relative heat-flow thermograms are separated for clarity.

FIG. 3 shows an X-ray powder diffractogram (XRPD) for MMC-APIs, at TO (i.e. after API loading).

FIG. 4 shows an X-ray powder diffractogram (XRPD) for MMC-APIs, after storage for 1 month (T1) at 75% relative humidity and room temperature.

FIG. 5 shows an X-ray powder diffractogram (XRPD) for MMC-APIs, after storage for 6 months (T6) and 8 Months (T8), or 12 months (T12) at 75% relative humidity and room temperature.

### GENERAL METHODS FOR PREPARATION

### I. General Method for the Preparation of Particulate Anhydrous Mesoporous Magnesium Carbonate (MMC)

A particulate anhydrous and substantially amorphous mesoporous magnesium carbonate (MMC) as disclosed and claimed herein, may be prepared by a process comprising the steps of:
(i) stirring magnesium oxide (MgO) and methanol under a CO₂ pressure of 0.5-8 bar, and at a temperature of 15-60° C. for 12 hours-1 week in a pressure reactor, providing a reaction mixture;
(ii) heating the reaction mixture under a pressure of 100 mbar-1013 mbar, and at a temperature of −10-70° C. providing a gel;
(iii) drying the gel of step (ii) at 70-120° C., providing particles;
(iv) finally heat-treating the particles obtained in step (iii) at a temperature of up to 300° C.; and
(v) optionally milling and fractionating the particles obtained in step (iv) with regard to size.

The stirring in step (i) may be performed at a rotation speed of 10-500 rpm, and the ratio MgO [g]/Methanol [ml] may be 1:12.5, i.e. 1.0 g MgO/12.5 ml methanol.

The final optional heat-treatment (iv) of the particles may be performed by using a furnace with normal atmosphere. The temperature may be ramped from room temperature and up to 300° C. for 5-15 hours, and the temperature may thereafter be fixed at the elevated temperature for up to 24 hours.

The obtained material is a solid material or a cake which is then crushed into a particulate material. This may be done by grinding or milling (e.g. jet milling). Particles of the desired size are then size fractionized in order to provide a particle size distribution exhibiting a d₁₀ value of 70-430 μm.

The fractionation step (v) with regard to size, may be performed by dry sieving or by wet sieving. The particle size may also be controlled during the synthesis by using different types of reactors, raw material, or different methods for heat treatment.

### Material Characterization

Pore size is determined using nitrogen gas adsorption. Measurements are made on a Gemini VII 2390 or a Tristar® ∥ Plus 3030 surface area and porosity analyzer (Micromeritics, Norcross, GA, USA) operated at 77.3 K, providing data to be used for determining pore size, pore volume and BET surface area of MMC and MMC loaded with API (MMC-API), respectively). Prior to analysis, 100-200 mg sample is added to a sample tube and degassed without or under vacuum for at least 12 hours at 105° C. Pore size distributions are obtained using density functional theory (DFT) applied to the adsorption branch of nitrogen sorption isotherms. The surface area is determined by using well-recognized BET equation, and hence calculated from the nitrogen sorption isotherms (Brunauer et al, JACS, 60, 1938, 309-319).

It is to be noted that the BET surface area, measured by nitrogen adsorption analysis as herein described, may be higher if measured on an MMC which has not undergone heat treatment as herein described. Heat treating the MMC, i.e. exposing the MMC to elevated temperatures for a prolonged time, such as above 200° C. for over 10 hours in an oven, reduces the residual methanol content to typically below 4% by weight. Residual methanol is mostly dispersed inside the pores of the MMC which, depending on the amount by weight, may impact measurements of BET surface area by nitrogen adsorption analysis. The BET surface area of MMC which has not been heat treated may thus vary from 400-900 m²/g (such as 450-900 m²/g or 500-850 m²/g). The BET surface area as measured in accordance with the present invention, is measured on heat treated MMC.

Powder XRPD patterns may be obtained on a Bruker D8 Advance Twin-Twin diffractometer (Bruker UK Ltd., Coventry, UK) with Cu-K_{α} radiation (λ=1.54 Å), generating XRPD patterns through elastic X-ray scattering. Prior to the analysis, samples are ground, dispersed with ethanol and applied as a thin layer upon a zero-background silicon sample holder, or as a dry powder. Any residual solvent is evaporated under a heat lamp prior to analysis. The analysis setup may be in the 20 range 20-80 degrees, 5-80 degrees or 5-65 degrees.

Presence or non-presence of crystals as detected by DSC is determined by equilibrating a weighed sample in a DSC (Differential Scanning calorimetry) sample holder at a suitable temperature. The temperature is ramped at 10° C./min to a suitable temperature at which the sample is kept isothermally for 5 minutes before ramping the temperature down to the equilibration temperature. Finally, the temperature is ramped to well above the melting point of the sample. The equilibration temperature may be −35° C. and the isothermal temperature may be 80° C.

The particle size distributions are measured using laser diffraction with the Malvern Mastersizer 3000, using a dry method. The light scattering data, converted to particle size distribution are analyzed using Mie-scattering model, using the non-spherical particle type and MMC (MgCO₃) as material (i.e. MgCO₃ settings for the refractive index, adsorption index and density). Prior to adding the sample to the instrument, the sample container is mixed well in order to ensure good sampling. A few grams of powder is added to instrument for the measurement, the measurement time is set to 10-30 seconds. The lower obstruction limit is set to 0.5% and the upper limit to 5%, the air pressure is set to 1.5 barg. During the measurement the feed rate is constantly adjusted so that the obstruction is kept between 0.5% and 5%. All measurements are run in at least triplicate, from which an average result is calculated.

The particle size distribution of an MMC-API, or MMC, may also be measured using a wet method by laser diffraction with the Malvern Mastersizer 3000 with a Hydro MV accessory. The light scattering data, converted to particle size distribution are analyzed using Mie-scattering model, using the non-spherical particle type. The refractive index was set to 1.72 and absorption 0.01. In the software the dispersant is set as water with a refractive index of 1.33 and a level sensor threshold of 100. Maximum pump speed (3500 rpm) is used to prevent sedimentation of dispersed MMC-API, or MMC. All of the measurements including MMC-API, or MMC, are done in 10 mM NaOH. A background is taken with the cell filled with 10 mM NaOH and 3500 rpm pumping. A typical analysis includes 20 mg of MMC-API, or MMC, dispersed in 2.5 ml 10 mM sodium hydroxide in a 5 ml glass vial by 2 minutes of bath sonication. After sonication the MMC-API, or MMC, the sample is transferred to the measurement cell. The vial is rinsed several times to make sure all of the particular material has been transferred. Measurement duration is 10 seconds background and 10 seconds sample. Six sub runs are made, upon which an average result is calculated.

### Investigation of Powder Flowability

### Tapped Density and Bulk Density Measurement

A mechanically tapping device (Pharma Test PT-TD, Hainburg, Germany) is used to evaluate the powder's propensity to dense packing. A glass cylinder with a diameter of 12 mm (n=3) is filled with 10 ml of powder and weighed to obtain the initial bulk density, ρBulk. Thereafter, the cylinder is mechanically tapped with a constant velocity until the most stable arrangement is achieved and the volume does no longer decrease. A comparison between the ρBulk, and the final bulk density, ρtapped, is made. By measuring the untapped apparent volume, V₀, and the final tapped apparent volume, V_{f}, the compressibility index and Hausner ratio is calculated using equation 1 and 2. They are used as a measurement of the powder's flowability (European Pharmacopeia 9.0).

$$Compressibility Index ( \% ) = 100 \times ( {V}_{0} - {V}_{f} / {V}_{0} ) \qquad Equation 1$$

$$Hausner ratio = {V}_{0} / {V}_{f} \qquad Equation 2$$

### II. Preparation of a Solid Substantially Amorphous Api (MMC-API)

A solid substantially amorphous active pharmaceutical ingredient, comprising an API in admixture with particulate anhydrous and substantially amorphous mesoporous magnesium carbonate (MMC), may be prepared by
a. dissolving an API in a solvent such as an organic solvent;
b. adding a particulate anhydrous and substantially amorphous mesoporous magnesium carbonate (MMC) as herein disclosed and claimed, to the API solution of step (a);
c. evaporating the solvent; and
d. optionally drying the final product.

The API starting materials apremilast, binimetinib, cabozantinib, eliglustat, enzalutamide, etravirine, idelalisib, tofacitinib and vandetanib were all purchased from Chemtronica AB, Sollentuna, Sweden.

The ratio API [g]/anhydrous and particulate substantially amorphous mesoporous magnesium carbonate (MMC) [g] depends on the target API load: e.g. 2/8 for 20 wt % API load, or 3/7 for 30 wt % API load.

Examples of solvents useful in dissolving the API in step a) are lower alcohols such as methanol or ethanol, and acetone or mixtures thereof. Also 1-butanol, 2-butanol, acidified ethanol (0.1% 1M HCl), butyl acetate, tert-butylmethyl ether, dichloromethane, dimethyl sulfoxide, ethyl acetate, ethyl ether, ethyl formate, formic acid, heptane, isobutyl acetate, isopropyl acetate, methyl acetate, 3-methyl-1-butanol, methylethyl ketone, 2-methyl-1-propanol, pentane, 1-pentanol, 1-propanol, 2-propanol, propyl acetate, and triethylamine may be useful in dissolving an API.

Evaporation of the solvent in step c) may be performed at a reduced pressure, and at a temperature of room temperature and up to 70° C., such as 50-70° C.

The optional drying in step d) may be performed at a temperature of 70-100° C.

A high API load may be useful to reduce the amount of mesoporous material and in order to reduce the size of a capsule or tablet when formulating the amorphous API into a drug product, but the amount of API cannot be too high due to the risk of crystallization.

To obtain the substantially amorphous active pharmaceutical ingredient according to the present invention (MMC-API), the particulate anhydrous and substantially amorphous mesoporous magnesium carbonate (MMC) obtained after sieving, is mixed with a solution of an API in a container, such as an evaporation flask or round flask, or in a reactor, such as a glass/glass lined/stainless steel/Hastelloy reactor. The loading step is dependent on the properties of the active pharmaceutically ingredient.

API is added to a container whereupon an adequate amount of a suitable organic solvent is added in order to dissolve the API. The API-solvent mixture is sonicated at a temperature in the range from room temperature to 50° C., or as applicable, until the API is completely dissolved (typically 0-30 minutes).

Thereafter, MMC particles (particulate anhydrous and substantially amorphous mesoporous magnesium carbonate) prepared as described in the General Method I above (General Method for the preparation of particulate anhydrous Mesoporous Magnesium Carbonate (MMC)), is added to the API solution. The target API load is 20-30% by weight. The mesoporous particles (MMC) are mixed with the API solution at room temperature (20-25° C.) by swirling the flask by hand for 10 seconds whereupon the container is attached to a rotary evaporator (Rotavapor® R-300 with Heating Bath B-305, Heating Bath B-300 Base, Vacuum pump V-300 and Interface I-300 or

Interface I-300 Pro, Büchi, Flawil, Switzerland). Solvent is evaporated from the mixture at a pressure in the range from 100-650 mbar at a temperature in the range from room temperature to 65° C. and a rotation speed of 100 rpm. The solid substantially amorphous active pharmaceutical ingredient, herein called an MMC-API, obtained from solvent evaporation is thereafter placed in an oven for final drying typically at 80° C. for 20-24 hours. The dried MMC-API is transferred from the container and analyzed by nitrogen gas adsorption to determine pore size, pore volume and BET surface area, as well as XRPD and DSC to determine whether the API is present in its amorphous state.

Nitrogen gas adsorption is performed on a Tristar® II Plus 3030 surface area and porosity analyzer (Micromeritics, Norcross, GA, USA) operated at 77.3 K. 100-200 mg MMC-API is added to a sample tube and degassed under vacuum for at least 12 hours at 105° C. prior to analysis.

XRPD is measured using a Bruker D8 TwinTwin X-ray Diffractometer (Bruker UK Ltd., Coventry, UK) with Cu-K_{α} radiation (λ=1.54 Å). Prior to the analysis, MMC-APIs are ground using a mortar and a pestle, and the powder poured onto a silicon zero background sample holder with a cavity. The analysis set-up is in the 20 range 5-65 degrees.

DSC analysis is performed using a DSC Q2000 (TA Instruments, Newcastle, DE, USA). 2-6 mg of MMC-API is applied onto an aluminum pan, onto which an aluminum lid is placed and firmly closed using a crimper. To allow moisture from the sample to evaporate during the analysis, a pinhole is made in the middle of the pan using a needle.

The DSC analysis was run accordingly:

### Cycle 1:

Equilibration at −35° C.

### Cycle 2:

Ramp 10° C./min to 80.00° C.
Isothermal for 5 minutes.
Ramp 10° C./min to −35° C.

### Cycle 3:

Ramp 10° C./min to well above melting temperature of each respective API.

Solid substantially amorphous active pharmaceutical ingredients (MMC-APIs) are stored at room temperature in a desiccator containing a saturated NaCl solution, providing for a 75% relative humidity atmosphere. After 1 month of storage the MMC-APIs are analyzed with XRPD and DSC, as described above, to determine whether they are still amorphous or if they have crystallized.

The solid substantially amorphous active pharmaceutical ingredient (MMC-API) according to the present invention, may also be obtained by first preparing a solution of the API in a suitable solvent followed by wet impregnation onto the MMC, by spray-drying the dissolved API together with the dispersed MMC particles, by spraying the API onto MMC material suspended by a gas-stream (fluid-bed setup), by low- or high-shear wet granulation whereby the dissolved API may be applied by spraying, or by any other pharmaceutical process method.

If the admixture of MMC and API (i.e. the substantially amorphous active pharmaceutical ingredient according to the present invention, the MMC-API) is prepared on MMC which has not been heat treated, i.e. an MMC having a residual methanol content typically above 7 wt %, it is to be noted that the BET surface area may be higher on such MMC-API. The BET surface area of an MMC-API prepared on an MMC that has not been heat treated may in such case thus vary from 300-600 m²/g (such as 310-550 m²/g or 320-500 m²/g). The BET surface area as described and claimed in accordance with the present invention, is measured on MMC-API which has been prepared on MMC that was heat treated prior to API loading into the MMC material.

### Stability Testing

A solid substantially amorphous active pharmaceutical ingredient as disclosed and claimed herein (MMC-API), is stored at room temperature in a desiccator containing a saturated NaCl mixture so that the relative humidity is substantially 75%. The stability of the amorphous form is measured using XRPD and DSC at different time points such as 1 month, 1 year or longer.

Powder XRPD patterns are obtained on a Bruker D8 Advance Twin-Twin diffractometer (Bruker UK Ltd., Coventry, UK) with Cu-K_{α} radiation (λ=1.54 Å), generating XRPD patterns through elastic X-ray scattering. Prior to the analysis, samples may be ground, dispersed with ethanol and applied as a thin layer upon a zero-background silicon sample holder, or as a dry powder. Any remaining solvent is evaporated, such as under a heat lamp or infrared light, prior to the analysis. The analysis setup may be in the 20 range 20-80 degrees, 5-80 degrees or 5-65 degrees.

DSC analysis is determined by equilibrating a weighed sample in a DSC (Differential Scanning calorimetry) sample holder at a suitable temperature. The temperature is ramped at 10° C./min to a suitable temperature at which the sample is kept isothermally for 5 minutes before ramping the temperature down to the equilibration temperature. Finally, the temperature is ramped to a temperature well above the melting point of the API. The equilibration temperature may be −35° C. and the isothermal temperature may be 80° C.

To assess the chemical integrity of the APIs and excipients, in case excipients are used, a High Performance Liquid Chromatography (HPLC) system, with the appropriate software and equipped with suitable pump, auto-sampler, column, column oven and UV-VIS detector may be used. The analytical column used for the separation is selected considering the type of system that is used and the chemical entity that is analyzed. A typical analysis is performed, but not limited to, under constant column temperature of 25±2° C. and the separation is typically, but not limited to, carried out in isocratic mode with mobile phase constituting acetonitrile. Prior to use, the mobile phase may be filtered using millipore 0.45 μm filter and degassed on an ultrasonic bath. After optimization, the ideal flow rate is identified and samples of suitable volume and concentration is injected into the HPLC system to initiate the analysis. The analytical goal is to identify the parent chemical entity and/or the absence or presence of any chemical degradation products.

### Pharmaceutical Formulations

A solid substantially amorphous active pharmaceutical ingredient as herein described and claimed (MMC-API), may be formulated as an oral pharmaceutical formulation in admixture with a pharmaceutically and pharmacologically acceptable excipient, carrier and/or diluent. Examples of a useful oral pharmaceutical formulation (a drug product) may be selected from any one of a tablet, a powder, a capsule, with solid substantially amorphous API, a granule or a cachet, each containing a predetermined amount of an amorphous API as herein described and claimed.

Examples of pharmaceutically acceptable excipients, carriers and/or diluents useful when formulating a solid substantially amorphous active pharmaceutical ingredient as herein described and claimed (MMC-API), are thickeners, flavoring agents, diluents, emulsifiers, dispersing aids, carrier substances, lubricants or binders. Typical pharmaceutical carriers include, but are not limited to, binding agents (e.g. pregelatinized maize starch, polyvinylpyrrolidone or hydroxypropyl methylcellulose, etc.); fillers (e.g. lactose, glucose, sucrose and other sugars, microcrystalline cellulose, pectin, gelatin, calcium sulfate, ethyl cellulose, polyacrylates or calcium hydrogen phosphate, etc.); lubricants (e.g. magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g. starch, and sodium starch glycolate); wetting agents; diluents; coloring agents; emulsifying agents; pH buffering agents; preservatives; and mixtures thereof.

A substantially amorphous active pharmaceutical ingredient as disclosed and claimed herein (MMC-API), may be formulated into tablets, powder, capsules or cachet, using any suitable formulation technique known to a skilled person. Alternatively, the substantially amorphous active pharmaceutical ingredient (MMC-API) may be filled into a capsule, such as a hard gelatin capsule or a soft gelatin capsule.

### Medical Use

One aspect of the present invention is the use of a solid substantially amorphous active pharmaceutical ingredient (MMC-API), as herein disclosed and claimed, in therapy.

Yet an aspect of the invention is a solid substantially amorphous active pharmaceutical ingredient as herein disclosed and claimed (MMC-API), for the treatment of any medical condition selected from psoriasis such as psoriatic arthritis or plaque psoriasis; cancer such as melanoma including unresectable or metastatic melanoma; or NSCLC; cell carcinoma such as advanced renal cell carcinoma (RCC); Gaucher 4 disease type 1 (GD1); prostate cancer such as metastatic castration-resistant prostate cancer; Human Immunodeficiency Virus infections (HIV) such as HIV Type 1 (HIV 1); diabetes; pulmonary arterial hypertension (PAH); coronavirus disease 2019 (COVID-19);

Leukemia such as Chronic Lymphocytic Leukemia (CLL); Follicular B-Cell non-Hodgkin Lymphoma (FL); Small Lymphocytic Lymphoma (SLL); Rheumatoid Arthritis; and thyroid cancer such as symptomatic or progressive medullary thyroid cancer.

Yet an aspect of the invention, is the use of a solid substantially amorphous active pharmaceutical ingredient as herein disclosed and claimed (MMC-API), for the manufacture of a medicament for the treatment of any medical condition selected from psoriasis such as psoriatic arthritis or plaque psoriasis; cancer such as melanoma including unresectable or metastatic melanoma; or NSCLC; cell carcinoma such as advanced renal cell carcinoma (RCC); Gaucher 4 disease type 1 (GD1); prostate cancer such as metastatic castration-resistant prostate cancer; Human Immunodeficiency Virus infections (HIV) such as HIV Type 1 (HIV 1); diabetes; pulmonary arterial hypertension (PAH); coronavirus disease 2019 (COVID-19); Leukemia such as Chronic Lymphocytic Leukemia (CLL); Follicular B-Cell non-Hodgkin Lymphoma (FL); Small Lymphocytic Lymphoma (SLL); Rheumatoid Arthritis; and thyroid cancer such as symptomatic or progressive medullary thyroid cancer.

Yet an aspect of the invention, is a method for the treatment of any medical condition selected from psoriasis such as psoriatic arthritis or plaque psoriasis; cancer such as melanoma including unresectable or metastatic melanoma; or NSCLC; cell carcinoma such as advanced renal cell carcinoma (RCC); Gaucher 4 disease type 1 (GD1); prostate cancer such as metastatic castration-resistant prostate cancer; Human Immunodeficiency Virus infections (HIV) such as HIV Type 1 (HIV 1); diabetes; pulmonary arterial hypertension (PAH); coronavirus disease 2019 (COVID-19); Leukemia such as Chronic Lymphocytic Leukemia (CLL); Follicular B-Cell non-Hodgkin Lymphoma (FL); Small Lymphocytic Lymphoma (SLL); Rheumatoid Arthritis; and thyroid cancer such as symptomatic or progressive medullary thyroid cancer; whereby a solid substantially amorphous active pharmaceutical ingredient as herein disclosed and claimed (MMC-API), is administered to a subject in need of such treatment.

The use or treatment of the medical indications disclosed herein, may be monotherapy, or combination therapy with for example a drug used as standard of care therapy.

### EXAMPLES

### Example 1—Preparation and Flowability of Particulate Anhydrous and Substantially Amorphous Mesoporous Magnesium Carbonate (MMC)

### Particulate Anhydrous and Substantially Amorphous Mesoporous Magnesium Carbonate Batch 1 was Prepared by:

(i) stirring 2000 g magnesium oxide (MgO) (PharMagnesia® MO Type B150 purchased from Lehmann & Voss & Co. KG, Hamburg) and 25 L of methanol (purchased from Solveco AB, Rosersberg, Sweden) in a conical dryer;
(ii) applying 4 bar CO₂ pressure and stirring (25 rpm) the solution from (i) at room temperature for 3 days;
(iii) releasing the pressure of the reaction liquid formed in (ii);
(iv) heating the reaction liquid from room temperature to 50° C. for 9 hours at 20 rpm. The temperature was hold at 50° C. for 7.5 hours, providing a gel;
(v) drying the gel at 50-160° C. for 7 hours at 950 mbar and 20 rpm. The temperature was hold at 160° C. for 16 hours, forming particles;
(vi) finally heat treating the particles of step (v) at a temperature of 250° C. in normal atmosphere using a furnace. The temperature was ramped from room temperature to 250° C. during 10 hours and hold at 250° C. for 10 more hours;
(vii) the obtained anhydrous and substantially amorphous mesoporous particulate magnesium carbonate was ground with a mortar and pestle and sieved (dry) (30 minutes, amplitude 100%) using a Vibratory Sieve Shaker AS 200 basic (Retsch GmbH, Haan, Germany) and sieves to mesh size 150 μm and 250 μm.

### Particulate Anhydrous and Substantially Amorphous Mesoporous Magnesium Carbonate Batch 2 was Prepared by:

(i) stirring 160 g magnesium oxide (MgO) (PharMagnesia® MO Type B150 purchased from Lehmann & Voss & Co. KG, Hamburg) and 2 L of methanol (purchased from Solveco AB, Rosersberg, Sweden) in a stainless steel pressure reactor;
(ii) applying 4 bar CO₂ pressure and stirring (400 rpm) the solution from (i) at room temperature for 7 days;
(iii) transferring the reaction liquid formed in (ii) to two evaporation flasks (a and b) connected to a rotary evaporator;
(iv) heating the reaction liquids at 60° C. and at 60 rpm using the rotary evaporator for 6 hours, providing a gel;
(v)
   a. drying the gel at 70° C. for 1 hour, 80° C. for 30 minutes and 100° C. for 1 hour the rotary evaporator,
   b. drying the gel at 100° C. using the rotary evaporator for 3 hours, forming particles;
(vi) finally heat treating the particles of step (v) at a temperature of 250° C. in normal atmosphere using a furnace. The temperature was ramped from room temperature to 250° C. during 10 hours and hold at 250° C. for 10 more hours;
(vii) the obtained anhydrous and substantially amorphous mesoporous particulate magnesium carbonate was ground with a mortar and pestle and sieved (dry) (30 minutes, amplitude 100%) using a Vibratory Sieve Shaker AS 200 basic (Retsch GmbH, Haan, Germany) and sieves with mesh sizes: 1 mm, 710 μm, 500 μm, 250 μm and 150 μm.

Nitrogen gas adsorption analysis, XRPD, DSC and investigation of powder flowability, including tapped density and bulk density measurement, on the obtained MMC are performed as described elsewhere herein.

Particulate anhydrous and substantially amorphous mesoporous magnesium carbonate Batch 3 was prepared by repeating the method described for Batch 2 four times, yielding four sub batches, varying the CO₂ pressure between 4 and 4.5 bar and reaction time between 4 and 6 days (i). The reaction liquids were heated to 60° C. for 4.5-5 (iv) and further heated to between 100° C. and 105° C. for between 1 and 3 hours (v) b. After final heat treatment (vi) and sieving (vii) the sub batches were pooled as Batch 3.

### Results

### Batch 1

A fraction with a mean particle sizes (D₅₀) of 192.0 μm was obtained. The obtained particulate anhydrous and substantially amorphous mesoporous magnesium carbonate (MMC) had a BET surface area of 373 m²/g and a pore volume of 0.56 cm³/g with ˜100% of the pore volume from pores<10 nm in diameter.

### Batch 2

A fraction with a mean particle sizes (D₅₀) of 209.0 μm was obtained. The obtained particulate anhydrous and substantially amorphous mesoporous magnesium carbonate (MMC) had a BET surface area of 365 m²/g and a pore volume of 0.76 cm³/g with ˜100% of the pore volume from pores<10 nm in diameter.

### Batch 3

A fraction with a mean particle sizes (D₅₀) of 211.0 μm was obtained. The obtained particulate anhydrous and substantially amorphous mesoporous magnesium carbonate (MMC) had a BET surface area of 402 m²/g and a pore volume of 0.64 cm³/g with ˜100% of the pore volume from pores<10 nm in diameter.

The bulk and tapped density for the fractions are shown in Table 1. By using the obtained values, the Carr index and Hausner ratio were calculated for the fraction and the flow property classified according to European Pharmacopeia 9.0. The fraction was classified to good. The results are summarized in Table 1 below.

TABLE 1
Flowability properties of the fraction obtained in Example 1 of particulate
anhydrous and substantially amorphous mesoporous magnesium carbonate (abbreviated
MMC in the table below). Values are given as mean and (s.d).

| | | | | Bulk | Tapped | | | |
|---|---|---|---|---|---|---|---|---|
| | D₁₀ | D₅₀ | D₉₀ | density | density | Carr | Hausner | Flow |
| MMC | [μm] | [μm] | [μm] | [g/ml] | [g/ml] | index | ratio | property* |
| Batch 1 | 96.7 | 192.0 | 306.0 | 0.58 | 0.64 | 10.67 | 1.12 | Good |
| Batch 2 | 84.4 | 209.0 | 352.0 | 0.47 | 0.525 | 10 | 1.11 | Good |
| Batch 3 | 94.0 | 211.0 | 356.0 | 0.53 | 0.60 | 10.33 | 1.12 | Good |

*According to European Pharmacopeia 9.0 (2016), Chapter 2.9.36. Powder Flow

### Example 2—Preparation and Stability Testing of Solid Substantially Amorphous Idelalisib (i.e. MMC-Idelalisib)

By following the general procedure described above, the API idelalisib was loaded into MMC particles (particulate anhydrous and substantially amorphous mesoporous magnesium carbonate) prepared according to Example 1 above (MMC Batch 2 was used in this Example). The API was used in its free form.

1538.0 mg idelalisib was added to an evaporation flask. 200 ml acetone was added in order to dissolve the idelalisib. The mixture was sonicated for 1 minute at room temperature until the idelalisib was dissolved.

3524.2 mg of mesoporous particles (MMC) were added to the idelalisib solution. The target API load of idelalisib was 30 wt %. The mesoporous particles (MMC) were mixed with the idelalisib solution at room temperature (20-25° C.) by swirling the flask by hand for 10 seconds, whereupon the evaporation flask was attached to the rotary evaporator (with Interface 1-300 Pro). The solvent was evaporated from the mixture at 600 mbar, a temperature of 55° C. and a rotation speed of 100 rpm. After 2 min the pressure was increased to 650 mbar to minimize the risk of boiling. After 4 minutes the pressure was reduced to 600 mbar whereupon the evaporation continued for an additional 5 minutes. The pressure was further reduced to 500 mbar and the evaporation continued for 4 minutes. Lastly the pressure was reduced to 300 mbar for 1 minute until the acetone had evaporated and a solid, substantially amorphous idelalisib, herein called MMC-idelalisib, was obtained.

The MMC-idelalisib was put into an oven for final drying at 80° C. for 24 hours. The finally dried MMC-idelalisib was removed from the evaporation flask and analyzed by nitrogen gas adsorption to determine pore size, pore volume and BET surface area, as well as XRPD and DSC to determine whether idelalisib was present in its amorphous state.

Nitrogen gas adsorption was performed on a Tristar® II Plus 3030 surface area and porosity analyzer (Micromeritics, Norcross, GA, USA) operated at 77.3 K. 100-200 mg MMC-idelalisib was added to a sample tube and degassed under vacuum for at least 12 hours at 105° C. prior to analysis.

XRPD was measured using a Bruker D8 TwinTwin X-ray Diffractometer (Bruker UK Ltd., Coventry, UK) with Cu-K_{α} radiation (λ=1.54 Å). The MMC-idelalisib was ground and the powder poured onto a silicon zero background sample holder with a cavity. The analysis set-up was in the 20 range: 5-65 degrees.

DSC analysis was performed using a DSC Q2000 (TA Instruments, Newcastle, DE, USA). 3.52 mg of MMC-idelalisib was added to an aluminum pan, onto which an aluminum lid was placed and firmly closed using a crimper. To allow moisture from the sample to evaporate during the analysis, a pinhole was made in the middle of the pan using a needle.

### The DSC Analysis was Run Accordingly:

### Cycle 1:

Equilibration at −35° C.

### Cycle 2:

Ramp 10° C./min to 80.00° C.
Isothermal for 5 minutes.
Ramp 10° C./min to −35° C.

### Cycle 3:

Ramp 10° C./min to 300° C.

The MMC-idelalisib was stored at room temperature in a desiccator containing a saturated NaCl solution, resulting in a 75% relative humidity atmosphere. After 1 month of storage the MMC-idelalisib was analyzed with XRPD and DSC, according to described methods herein, to determine whether it was still amorphous or if it had crystallized.

The specific conditions given in this example for the loading of idelalisib into the particulate anhydrous and substantially amorphous mesoporous magnesium carbonate, is summarized in Table 2a and Table 2b.

### Example 3—Preparation and Stability Testing of Solid Substantially Amorphous APIs (i.e. MMC-API)

By following the general procedure described above and more specifically by following the specific procedure as described for Example 2 using the specific conditions presented in Table 2a and Table 2b below, the APIs etravirine, vandetanib, binimetinib, cabozantinib, tofacitinib, eliglustat, enzalutamide and apremilast, were each loaded into MMC particles (particulate anhydrous and substantially amorphous mesoporous magnesium carbonate) prepared according to Example 1.

All APIs were used in its free form.

All MMC-APIs were analyzed by nitrogen gas adsorption, XRPD and/or DSC as described above. The specific conditions for each API are presented in Table 2a and Table 2b below. All MMC-APIs were analyzed by nitrogen gas adsorption, XRPD and/or DSC as described above.

TABLE 2a
Selected APIs loaded into MMC, amounts and parameters applied
to produce the corresponding amorphous MMC-APIs.

| API in the | | API | | | Sonication | | |
|---|---|---|---|---|---|---|---|
| MMC-API | MMC | load | API | Solvent | [37 Hz] | | MMC |
| admixture | batch | [wt %] | [mg] | [ml] | [° C.] | [Min] | [mg] |
| Apremilast(a) | 1 | 30 | 297.3 | Acetone 150 | N/A | N/A | 699.5 |
| Apremilast(b) | 3 | 30 | 902.7 | Acetone 200 | N/A | N/A | 2106.6 |
| Binimetinib(a) | 2 | 30 | 310.8 | Acetone 500 | 40 | 30 | 701.2 |
| Binimetinib(b) | 3 | 30 | 908.25 | Acetone 1200 | 40 | 90 | 2100.75 |
| Cabozantinib(a) | 1 | 30 | 297.5 | Acetone 400 | 43-50 | 10 | 700.3 |
| Cabozantinib(b) | 3 | 20 | 401.4 | Acetone 200 | 30-40 | 14 | 1592.7 |
| Eliglustat(a) | 2 | 30 | 296.9 | Acetone 100 | 20 | 1 | 699.6 |
| Eliglustat(b) | 3 | 30 | 908.2 | Acetone 150 | 20 | 1 | 2103.2 |
| Enzalutamide(a) | 2 | 30 | 307.8 | Acetone 100 | 45 | 2 | 704.3 |
| Enzalutamide(b) | 3 | 25 | 502.7 | Acetone 100 | 40 | 15 | 1498.97 |
| Etravirine(a) | 2 | 20 | 209.8 | Acetone 250 | 40 | 15 | 802.0 |
| Etravirine(b) | 3 | 20 | 602.9 | Acetone 200 | 22-36 | 27 | 2401.9 |
| Idelalisib(a) | 2 | 30 | 1538.0 | Acetone 200 | RT | 1 | 3524.2 |
| Idelalisib(b) | 3 | 30 | 603.9 | Acetone 150 | 42 | 15 | 1402.5 |
| Tofacitinib(a) | 2 | 30 | 1521.3 | Acetone 200 | RT | 1 | 3527.0 |
| Tofacitinib(b) | 3 | 25 | 505.2 | Acetone 100 | 40 | 15 | 1501.5 |
| Vandetanib(a) | 2 | 20 | 401.41 | Ethanol 400 | 40 | 3 | 1601.3 |
| Vandetanib(b) | 3 | 20 | 302.8 | Ethanol 250 | 25 | 2 | 1201.7 |

TABLE 2b
Specific conditions for loading of APIs into MMC through solvent
evaporation according to the general loading description,
applied to produce the corresponding amorphous MMC-APIs.

| | | Solvent evaporation | | | |
|---|---|---|---|---|---|
| API in the | Rotation | Temper- | | | Final |
| MMC-API | speed | ature | Pressure | Duration | drying |
| admixture | [rpm] | [° C.] | [mbar] | [min] | [hours] |
| Apremilast(a) | 100 | 55 | 500 | 2 | 22 |
| | | | 450 | 2 | |
| | | | 300 | 3 | |
| | | | 200 | 4 | |
| | | | 100 | 2 | |
| Apremilast(b) | 100 | 55 | 520 | 0.33 | 24 |
| | | | 550 | 0.5 | |
| | | | 580 | 0.33 | |
| | | | 600 | 3 | |
| | | | 550 | 1.75 | |
| | | | 500 | 1.33 | |
| | | | 450 | 0.5 | |
| | | | 400 | 0.5 | |
| | | | 350 | 0.33 | |
| | | | 300 | 1 | |
| Binimetinib(a) | 100 | 55 | 550 | 15 | 24 |
| | | | 500 | 1 | |
| Binimetinib(b) | 100 | 55 | 550 | 2 | 21 |
| | | | 500 | 2 | |
| | | | 450 | 2 | |
| | | | 400 | 8 | |
| | | | 350 | 4 | |
| | | | 300 | 3 | |
| | | | 200 | 1 | |
| Cabozantinib(a) | 100 | 55 | 600 | 1 | 23 |
| | | | 500 | 1 | |
| | | | 450 | 2 | |
| | | | 400 | 3 | |
| | | | 300 | 0.33 | |
| | | | 350 | 3 | |
| | | | 250 | 2 | |
| | | | 200 | 12 | |
| | | | 100 | 3 | |
| Cabozantinib(b) | 100 | 55 | 500 | 3 | 21 |
| | | | 480 | 2.5 | |
| | | | 450 | 0.25 | |
| | | | 430 | 0.25 | |
| | | | 350 | 2 | |
| Eliglustat(a) | 100 | 55 | 500 | 2 | 24 |
| | | | 450 | 5 | |
| | | | 300 | 1 | |
| Eliglustat(b) | 100 | 55 | 500 | 2 | 21 |
| | | | 450 | 2 | |
| | | | 400 | 1 | |
| | | | 300 | 1 | |
| | | | 200 | 0.5 | |
| Enzalutamide(a) | 100 | 55 | 500 | 3 | 24 |
| | | | 450 | 1 | |
| | | | 400 | 2 | |
| | | | 300 | 1 | |
| | | RT | 250 | 1 | |
| Enzalutamide(b) | 100 | 55 | 500 | 3 | 22 |
| | | | 450 | 1.75 | |
| | | | 300 | 2 | |
| Etravirine(a) | 100 | 55 | 550 | 2 | 24 |
| | | | 500 | 2 | |
| | | | 450 | 5 | |
| | | | 400 | 10 | |
| | | RT | 300 | 2 | |
| Etravirine(b) | 100 | 55 | 500 | 3 | 24 |
| | | | 480 | 1 | |
| | | | 450 | 1 | |
| | | | 400 | 1 | |
| | | | 350 | 0.33 | |
| | | | 320 | 0.75 | |
| | | | 300 | 1 | |
| Idelalisib(a) | 100 | 55 | 600 | 2 | 24 |
| | | | 650 | 4 | |
| | | | 600 | 5 | |
| | | | 550 | 4 | |
| | | | 300 | 1 | |
| Idelalisib(b) | 100 | 55 | 500 | 2 | 22 |
| | | | 480 | 0.33 | |
| | | | 450 | 1 | |
| | | | 430 | 1 | |
| | | | 400 | 0.5 | |
| | | | 380 | 0.25 | |
| | | | 350 | 0.17 | |
| | | | 300 | 1 | |
| Tofacitinib(a) | 100 | 55 | 600 | 1 | 24 |
| | | | 650 | 5 | |
| | | | 600 | 4 | |
| | | | 550 | 3 | |
| | | | 300 | 1 | |
| Tofacitinib(b) | 100 | 55 | 500 | 2.17 | 22 |
| | | | 480 | 1.33 | |
| | | | 450 | 0.42 | |
| | | | 400 | 0.5 | |
| | | | 350 | 0.25 | |
| | | | 300 | 1 | |
| Vandetanib(a) | 100 | 65 | 500 | 1 | 20 |
| | | | 450 | 1 | |
| | | | 400 | 2 | |
| | | | 350 | 0.5 | |
| | | | 300 | 0.5 | |
| | | | 250 | 1 | |
| | | | 200 | 1 | |
| | | | 170 | 3 | |
| | | | 150 | 10 | |
| Vandetanib(b) | 100 | 60 | 550 | 0.5 | 24 |
| | | | 500 | 0.5 | |
| | | | 450 | 0.5 | |
| | | | 400 | 0.5 | |
| | | | 350 | 4 | |
| | | | 250 | 1 | |
| | | | 200 | 3 | |
| | | | 170 | 4 | |
| | | | 150 | 4 | |
| | | | 130 | 1 | |
| | | | 110 | 1 | |

### Results

All APIs were loaded into the particulate anhydrous and substantially amorphous mesoporous magnesium carbonate (MMC) as can be seen by the change in pore volume, BET surface area and pore size.

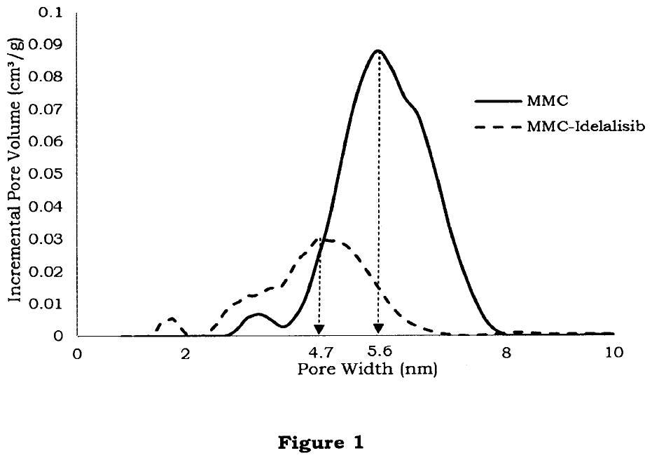

Results from nitrogen gas adsorption of unloaded mesoporous particulate magnesium carbonate (MMC) (Table 2c) and the material (MMC) after loading (Table 2d) are presented below. The peak pore width of MMC Batch 2 compared to idelalisib loaded into MMC Batch 2 (referred to as MMC-idelalisib) is illustrated in FIG. 1.

TABLE 2c
Pore volume, BET surface area and peak pore width of MMC.

| | Pore Volume | BET Surface | Peak Pore |
|---|---|---|---|
| | at p/p₀ 0.97 | Area | Width |
| MMC | [cm³/g] | [m²/g] | [nm] |
| Batch 1 | 0.56 | 373 | 4.7 |
| Batch 2 | 0.76 | 365 | 5.6 |
| Batch 3 | 0.64 | 402 | 4.9 |

TABLE 2d
Pore volume, BET surface area and peak pore width
of MMC-API, i.e. after loading of API into MMC.

| API in the | | Pore Volume | BET Surface | Peak Pore |
|---|---|---|---|---|
| MMC-API | API | at p/p₀ 0.97 | Area | Width |
| admixture | load | [cm³/g] | [m²/g] | [nm] |
| Apremilast(a) | 30 | 0.48 | 359 | 4.5 |
| Apremilast(b) | 30 | 0.35 | 302 | 4.2 |
| Binimetinib(a) | 30 | 0.38 | 255 | 5.2 |
| Binimetinib(b) | 30 | 0.44 | 373 | 4.5 |
| Cabozantinib(a) | 30 | 0.52 | 401 | 4.5 |
| Cabozantinib(b) | 20 | N/A | N/A | N/A |
| Eliglustat(a) | 30 | 0.30 | 187 | 4.8 |
| Eliglustat(b) | 30 | 0.22 | 198 | 4.1 |
| Enzalutamide(a) | 30 | 0.35 | 215 | 5.0 |
| Enzalutamide(b) | 25 | N/A | N/A | N/A |
| Etravirine(a) | 20 | 0.49 | 302 | 5.2 |
| Etravirine(b) | 20 | 0.51 | 333 | 4.9 |
| Idelalisib(a) | 30 | 0.40 | 313 | 4.7 |
| Idelalisib(b) | 30 | N/A | N/A | N/A |
| Tofacitinib(a) | 30 | 0.36 | 243 | 4.9 |
| Tofacitinib(b) | 25 | N/A | N/A | N/A |
| Vandetanib(a) | 20 | 0.61 | 327 | 5.4 |
| Vandetanib(b) | 20 | N/A | N/A | N/A |

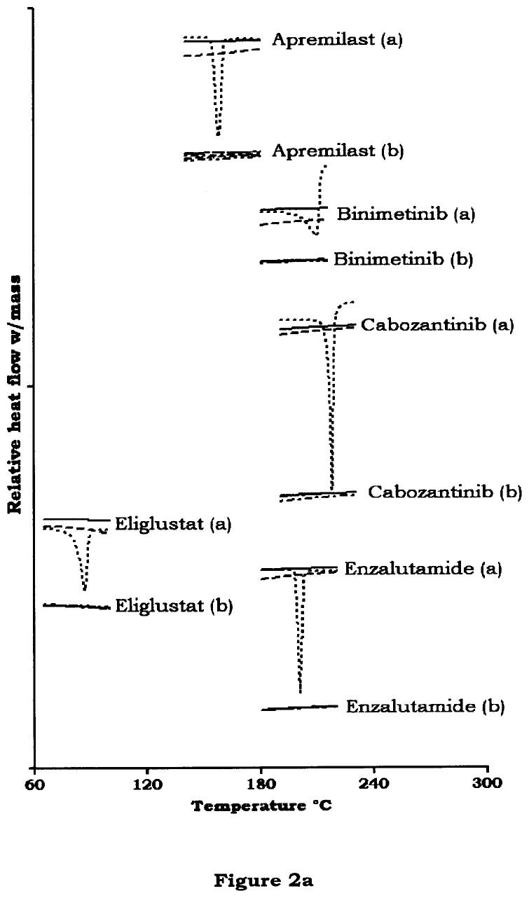

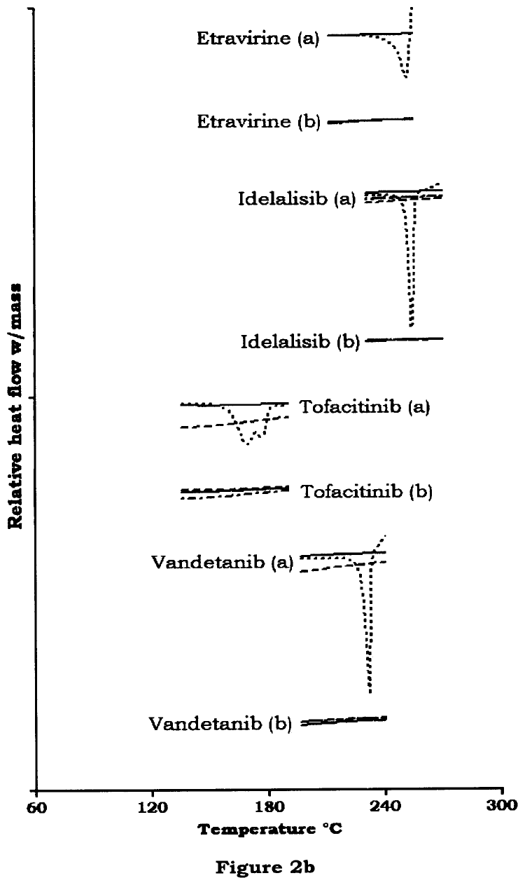

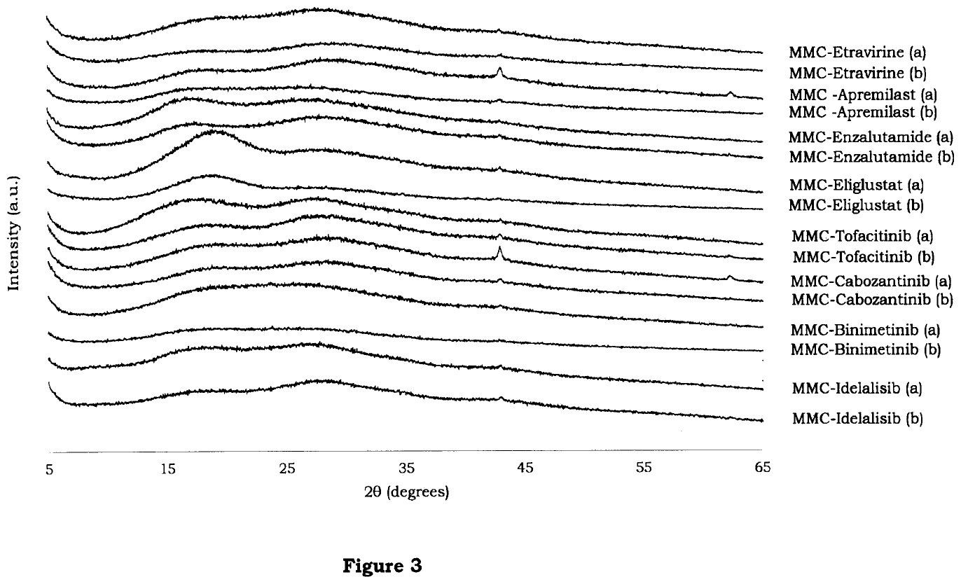

According to results from XRPD and/or DSC, all loaded APIs measured were amorphous, as shown in Table 2e, FIG. 2a, FIG. 2b and FIG. 3.

TABLE 2e
Physical state of API after loading into MMC (i.e. MMC-API).

| API in the | | | |
|---|---|---|---|
| MMC-API | | XRPD amorphous | DSC amorphous |
| admixture | API load | after loading | after loading |
| Apremilast(a) | 30 | Yes | Yes |
| Apremilast(b) | 30 | Yes | Yes |
| Binimetinib(a) | 30 | Yes | Yes |
| Binimetinib(b) | 30 | Yes | Yes |
| Cabozantinib(a) | 30 | Yes | Yes |
| Cabozantinib(b) | 20 | Yes | Yes |
| Eliglustat(a) | 30 | Yes | Yes |
| Eliglustat(b) | 30 | Yes | Yes |
| Enzalutamide(a) | 30 | Yes | Yes |
| Enzalutamide(b) | 25 | Yes | Yes |
| Etravirine(a) | 20 | Yes | Yes |
| Etravirine(b) | 20 | Yes | Yes |
| Idelalisib(a) | 30 | Yes | Yes |
| Idelalisib(b) | 30 | Yes | Yes |
| Tofacitinib(a) | 30 | Yes | Yes |
| Tofacitinib(b) | 25 | Yes | Yes |
| Vandetanib(a) | 20 | N/A | Yes |
| Vandetanib(b) | 20 | N/A | Yes |

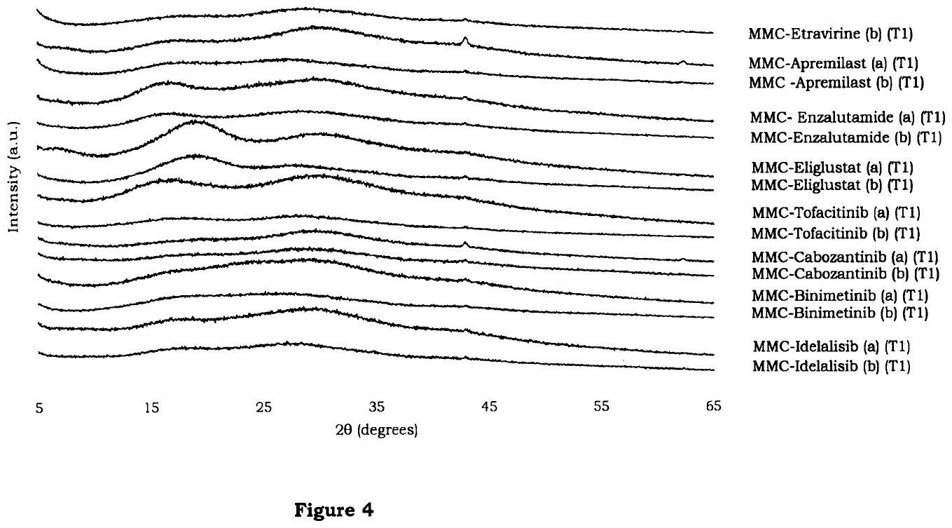

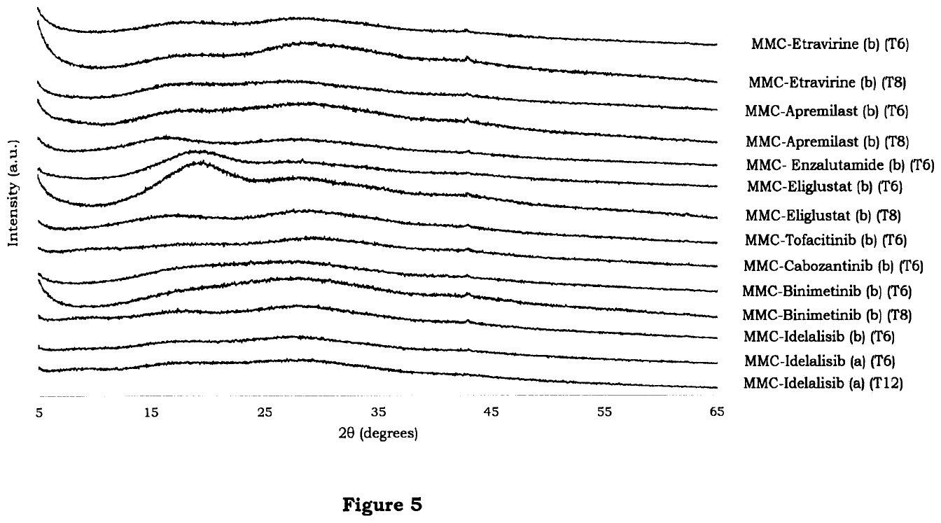

After storage for 1, 6 and 8 months, or for 12 months, at 75% relative humidity and room temperature, the MMC-APIs (except for etravirine) were analyzed with XRPD and DSC again. Results from XRPD and DSC are presented in Table 2f and illustrated in FIG. 2a, FIG. 2b, FIG. 3, FIG. 4, and FIG. 5.

TABLE 2f
Physical state of solid substantially amorphous API (i.e.
MMC-API) after storage for 1 month and/or 6 months,
at room temperature and 75% relative humidity. These
results are also presented in FIG. 4 and FIG. 5.

| | | XRPD | DSC | XRPD | DSC |
|---|---|---|---|---|---|
| | | amor- | amor- | amor- | amor- |
| | | phous | phous | phous | phous |
| API in the | | after | after | after | after |
| MMC-API | API | storage | storage | storage | storage |
| admixture | load | [1 month] | [1 month] | [6 months] | [6 months] |
| Apremilast(a) | 30 | Yes | Yes | N/A | N/A |
| Apremilast(b) | 30 | Yes | Yes | Yes⁽¹⁾ | Yes⁽¹⁾ |
| Binimetinib(a) | 30 | Yes | Yes | N/A | N/A |
| Binimetinib(b) | 30 | Yes | Yes | Yes⁽¹⁾ | Yes⁽¹⁾ |
| Cabozantinib(a) | 30 | Yes | Yes | N/A | N/A |
| Cabozantinib(b) | 20 | Yes | Yes | Yes | Yes |
| Eliglustat(a) | 30 | Yes | Yes | N/A | N/A |
| Eliglustat(b) | 30 | Yes | Yes | Yes⁽¹⁾ | Yes⁽¹⁾ |
| Enzalutamide(a) | 30 | Yes | Yes | N/A | N/A |
| Enzalutamide(b) | 25 | Yes | Yes | Yes | Yes |
| Etravirine(a) | 20 | N/A | N/A | N/A | N/A |
| Etravirine(b) | 20 | Yes | Yes | Yes⁽¹⁾ | Yes⁽¹⁾ |
| Idelalisib(a) | 30 | Yes | Yes | Yes⁽²⁾ | Yes⁽²⁾ |
| Idelalisib(b) | 30 | Yes | Yes | Yes | Yes |
| Tofacitinib(a) | 30 | Yes | Yes | N/A | N/A |
| Tofacitinib(b) | 25 | Yes | Yes | Yes | Yes |
| Vandetanib(a) | 20 | N/A | Yes | N/A | N/A |
| Vandetanib(b) | 20 | N/A | Yes | N/A | Yes |

⁽¹⁾Also amorphous after storage for 8 months
⁽²⁾Also amorphous after storage for 12 months

What is claimed:

1. A solid substantially amorphous active pharmaceutical ingredient, comprising an API in an amount of at least 20% by weight, in admixture with a particulate anhydrous and substantially amorphous mesoporous magnesium carbonate (MMC), wherein said admixture of MMC and API (MMC-API admixture) has:
(i) pores with a peak pore width in the range of 2 nm to 10 nm;
(ii) an average BET surface area in the range of 150-600 m²/g;
(iii) an average pore volume in the range of 0.1-1.2 cm³/g; and
(iv) an average particle size distribution exhibiting a d₁₀ value of 70-430 μm;
and wherein the API is selected from any one of apremilast, binimetinib, cabozantinib, eliglustat, enzalutamide, etravirine, idelalisib, tofacitinib and vandetanib

2. A solid substantially amorphous active pharmaceutical ingredient according to claim 1, wherein said solid substantially amorphous active pharmaceutical ingredient (MMC-API admixture) has pores with a peak pore width in the range of 3 nm to 9 nm.

3. A solid substantially amorphous active pharmaceutical ingredient according to claim 1, wherein said solid substantially amorphous active pharmaceutical ingredient (MMC-API admixture) has an average BET surface area in the range of 150-500 m²/g.

4. A solid substantially amorphous active pharmaceutical ingredient according to claim 1, wherein said solid substantially amorphous active pharmaceutical ingredient (MMC-API admixture) has an average pore volume in the range of 0.1-0.9 cm³/g.

5. A solid substantially amorphous active pharmaceutical ingredient according to claim 1, wherein said solid substantially amorphous active pharmaceutical ingredient (MMC-API admixture) has an average particle size distribution exhibiting a d₁₀ value of 70-350 μm.

6. A solid substantially amorphous active pharmaceutical according to claim 1, wherein said solid substantially amorphous active pharmaceutical ingredient (MMC-API admixture) has an average particle size distribution exhibiting a d₅₀ value of 75-500 μm.

7. A solid substantially amorphous active pharmaceutical ingredient according to claim 1, wherein said solid substantially amorphous active pharmaceutical ingredient (MMC-API admixture) has an average particle size distribution exhibiting a d₅₀ value of 75-400 μm.

8. A solid substantially amorphous active pharmaceutical ingredient according to claim 1, wherein said solid substantially amorphous active pharmaceutical ingredient (MMC-API admixture) has an average particle size distribution exhibiting a d₉₀ value of 170-500 μm.

9. A solid substantially amorphous active pharmaceutical ingredient according to claim 1, wherein said solid substantially amorphous active pharmaceutical ingredient (MMC-API admixture) has an average particle size distribution exhibiting a d₉₀ value of 220-500 μm.

10. A solid substantially amorphous active pharmaceutical ingredient according to claim 1, having a compressibility index of 15 or less.

11. A solid substantially amorphous active pharmaceutical ingredient according to claim 1, having a Hausner ratio of 1.18 or less.

12. An oral pharmaceutical formulation, comprising a solid substantially amorphous active pharmaceutical ingredient according to claim 1, in admixture with a pharmaceutically and pharmacologically acceptable excipient, carrier and/or diluent.

13. An oral pharmaceutical formulation which is bioequivalent to a formulation according to claim 12.

14. A method for treatment of cancer whereby a solid substantially amorphous active pharmaceutical ingredient according to claim 1 is administered to a subject in need of such treatment.

15. A method for treatment according to claim 14, wherein the cancer is leukemia.

16. A method for treatment according to claim 15, wherein the leukemia is Chronic Lymphocytic Leukemia (CLL).

17. A solid substantially amorphous active pharmaceutical ingredient according to claim 2, wherein said solid substantially amorphous active pharmaceutical ingredient (MMC-API admixture) has pores with a peak pore width in the range of 3 nm to 8 nm.

18. A solid substantially amorphous active pharmaceutical ingredient according to claim 2, wherein said solid substantially amorphous active pharmaceutical ingredient (MMC-API admixture) has pores with a peak pore width in the range of 3 nm to 7 nm.

19. A solid substantially amorphous active pharmaceutical ingredient according to claim 3, wherein said solid substantially amorphous active pharmaceutical ingredient (MMC-API admixture) has an average BET surface area in the range of or 60-430 m²/g.

20. A solid substantially amorphous active pharmaceutical ingredient according to claim 4, wherein said solid substantially amorphous active pharmaceutical ingredient (MMC-API admixture) has an average pore volume in the range of or 0.1-0.8 cm³/g.
